(19) United States
(12) Patent Application Publication
(10) Pub. No.: US 2023/0045427 A1
(43) Pub. Date: Feb. 9, 2023

(54) SENSOR FOR DETECTING A BIOANALYTE AND A METHOD FOR THE DETECTION THEREOF

(71) Applicant: Royal Melbourne Institute of Technology, Melbourne, Victoria (AU)

(72) Inventors: Taimur AHMED, Melbourne (AU); Mortiz DOERING, Leutkirch (DE); Ganganath PERERA, Melbourne (AU); Sumeet WALIA, Melbourne (AU); Madhu BHASKARAN, Melbourne (AU); Sharath SRIRAM, Melbourne (AU)

(21) Appl. No.: 17/757,700

(22) PCT Filed: Dec. 18, 2020
(86) PCT No.: PCT/AU2020/051396

§ 371 Date: Jun. 17, 2022

(30) Foreign Application Priority Data
Dec. 20, 2019 (AU) 2019904865

(51) Int. Cl. G01N 27/327; G01N 33/543; G01N 33/563; G01N 33/552

(52) U.S. Cl. CPC G01N 27/3275; G01N 33/5438; G01N 33/563; G01N 33/552

(57) ABSTRACT

The present invention provides a sensor for detecting a bioanalyte, comprising:
a substrate;
a pair of terminal electrodes disposed on the substrate in mutually spaced apart and opposing relation; and
a non-insulating sensing element applied to a surface of the substrate, between and in electrical contact with the pair of terminal electrodes wherein the sensing element provides a conduction path between the terminal electrodes, wherein the sensing element comprises an oxygen-deficient metal oxide layer and a bioanalyte binding site, and wherein when a voltage is applied across the sensor, an electrical signal is generated that is proportional to a change in conductance of the sensing element corresponding to binding of a bioanalyte to the bioanalyte binding site.

### FIELD OF THE INVENTION

The present invention relates to sensors and, in particular, to a non-invasive sensor for detecting a bioanalyte in a bodily fluid and a method for the detection thereof.

The invention has been developed primarily for use in detecting a range of bioanalytes in a bodily fluid and will be described hereinafter with reference to this application.

The following discussion of the background to the invention is intended to facilitate an understanding of the invention. However, it should be appreciated that the discussion is not an acknowledgement or admission that any of the material referred to was published, known or part of the common general knowledge in Australia or any other country as at the priority date of any one of the claims of this specification.

### BACKGROUND OF THE INVENTION

There are two approaches used to monitor/measure the level of target biomarkers (hereinafter, bioanalytes) in a tissue and/or biofluid. The first approach relies on the use of invasive sensors where the components of the sensor are directly in contact with tissues or bodily fluids that can cause infection, tissue damage and discomfort. The second approach relies on the use of non-invasive sensors, which employ different technologies to determine the level of bioanalytes in bodily fluids, including optical absorption, electrochemical, transduction and conductometry.

In terms of the non-invasive sensors, optical absorption-based non-invasive sensors are not particularly precise due to a close overlap of the weakly absorbing bands of the various bioanalytes that might be present in a bodily fluid, as well as the temperature sensitivity of such assays.

Electrochemical sensors, on the other hand, are more precise and therefore currently dominate the biosensing field. Such sensors operate by measuring the electrical signal generated by the reaction of a bioanalyte of interest with a sensing element associated with the sensor, where the generated electrical signal is proportional to the concentration of the bioanalyte. This electrochemical reaction in the sensor functions by eliciting a measurable current (amperometric), a measurable charge accumulation or potential (potentiometric), modifying conductive properties of a medium (conductometric) or impedimetric, by measuring resistance and reactance which combine to form impedance.

A sensor using electrochemical transduction typically requires a working electrode, a counter (or auxiliary) electrode and a reference electrode. The reference electrode is maintained at a distance from the site of the biological recognition element and analyte interaction to establish a known and stable potential. The working electrode acts as the transduction component when the interaction occurs whereas the counter electrode measures current and facilitates delivery of electrolytic solution to allow current transfer to the working electrode.

Conductometric sensors also rely on the use of electrodes to measure the ability of a medium to conduct an electrical current therebetween. However, conductometric sensors do not need the use of a reference electrode. These sensors also operate at low-amplitude alternating voltage, thus preventing Faraday processes on electrodes, and can be miniaturized and integrated easily using thin-film techniques.

While conductometric sensors have certain benefits, the sensitivity of such sensors is often hindered by using a polymer as the sensing element, which often leads to the sensor exhibiting poor durability and poor long-term stability.

As an alternative to a direct conductometric sensor, sensors based on field-effect transistors have also been developed. Field-effect transistors are devices with three terminals namely a source, a gate and a drain. These devices work as changes in the gate lead to a field effect which alters the conductivity between the source and the drain.

For example US 2010/2016256 describes a biosensor comprising: a substrate, a source electrode on the substrate, a drain electrode on the substrate, and at least one functionalized nanobelt on a surface of the substrate between the source electrode and the drain electrode, wherein the functionalized nanobelt has a chemically functionalized surface linked to one or more detector molecules for binding with a biological analyte to be detected such that an electric field gating effect is generated by binding of the analyte to the one or more detector molecules linked to the nanobelt surface. This device works as the binding of the molecule modifies the field effect of the nanobelt (the gate) such that the conductivity of the pathway between the source and the drain is modified and the change in conductivity can be monitored. In general, devices of this type suffer from two drawbacks.

Firstly, field-effect transistors are typically devices that turn on and off and have a non-linear response. In these devices the resistance will not change in a straight line as they typically have a small region of linear response and which then plateaus which means the device is difficult to use over a broad range of conditions.

Secondly, as will be appreciated by a skilled worker, in order for such a device to operate as described it is necessary that there is an insulating (dielectric) layer between the conduction path between the source and drain and the source of the gate bias (in this case the nanobelt). Devices of this type therefore suffer the disadvantage that they are relatively complicated to fabricate due to the number of different structural elements and are thus harder to produce on an industrial scale than sensors that are more structurally elegant.

The present invention seeks to provide a sensor for use in detecting a bioanalyte and a method for the detection thereof, which will overcome or substantially ameliorate at least some of the deficiencies of the prior art, or to at least provide an alternative.

### SUMMARY OF THE INVENTION

According to a first aspect of the present invention, there is provided a sensor for detecting a bioanalyte, comprising: a substrate; a pair of terminal electrodes disposed on the substrate in mutually spaced apart and opposing relation; and a non-insulating sensing element applied to a surface of the substrate, between and in electrical contact with the pair of terminal electrodes wherein the sensing element provides a conduction path between the terminal electrodes, wherein the sensing element comprises an oxygen-deficient metal oxide layer and an analyte binding site, and wherein when a voltage is applied across the sensor, an electrical signal is generated that is proportional to a change in conductance of the sensing element corresponding to binding of the analyte to the analyte binding site.

Preferably, the oxygen-deficient metal oxide layer is formed from a metal oxide selected from the group consisting of zinc oxide (ZnO), strontium titanium oxide (STO), tin oxide and titanium dioxide.

In one embodiment, the oxygen-deficient metal oxide layer has a thickness that falls within a range of about 50 nm to about 200 μm.

Preferably, the oxygen-deficient metal oxide layer is applied to the substrate surface by a technique selected from the group consisting of reactive sputtering, physical vapour deposition (PVD), chemical vapour deposition (CVD), metal organic chemical vapour deposition (MOCVD), pulsed laser deposition (PLD) and molecular beam epitaxy (MBE).

Preferably, the bioanalyte binding site is anchored to the oxygen-deficient metal oxide layer via an intermediate layer physically or chemically adsorbed to the oxygen-deficient metal oxide layer.

In one embodiment, the intermediate layer is produced by silanization of the oxygen-deficient metal oxide layer with a silanizing agent having a terminal functionality that is selected from the group consisting of an epoxy group, a thiol group, an amino group, a carboxy group and a hydroxy group.

In one embodiment, the silanizing agent is selected from the group consisting of (3-glycidyloxypropyl)trimethoxysilane, (3-mercaptopropyl)trimethoxysilane (MTS), (3-aminopropyl)triethoxysilane (APTES), and N-(2-aminoethyl)-3-aminopropyl-trimethoxysilane (AEAPTS).

In one embodiment, the oxygen-deficient metal oxide layer has a conductance that falls within a range of about 0.08 siemens/m² to about 0.6 siemens/m².

Preferably, the bioanalyte binding site is a biomolecule.

Suitably, the biomolecule is a protein, peptide, a lipo-peptide, a protein-binding carbohydrate or a protein-binding ligand.

In one embodiment, the biomolecule is a capture protein.

Suitably, the capture protein is a protein-binding scaffold, a T-cell receptor, a binding-fragment of a TCR, a variable lymphocyte receptor, an antibody and/or a binding-fragment of an antibody.

Preferably, the protein-binding scaffold is selected from the group consisting of: Adnectins, Affilins, Affibodies, Affimer molecules, Affitins, Alphabodies, Aptamers, Anticalins, Armadillo repeat protein-based scaffolds, Atrimers, Avimers, Designed Ankyrin Repeat Proteins (DARPins), Fynomers, Inhibitor Cystine Knot (ICK) scaffolds, Kunitz Domain peptides, Monobodies and/or Nanofitins.

Preferably, the binding-fragment of an antibody includes a Fab, (Fab′)₂, Fab′, single-chain variable fragment (scFv), di- and tri-scFvs, single domain antibodies (sdAb), Diabodies or a fusion protein including a binding-domain of an antibody.

In one embodiment, the bioanalyte binding site binds interleukin-6 (IL-6).

In one embodiment, the bioanalyte binding site binds C-reactive protein (CRP).

Preferably, the substrate is manufactured from a material selected from the group consisting of a silicon wafer, a polymer, a glass and a ceramic.

Suitably, the polymer is selected from the group consisting of polydimethylsiloxane (PDMS), polyimide (PI) and polyethylene naphthalate (PEN).

Suitably, the ceramic is selected from the group consisting of aluminium oxide (Al₂O₃), sapphire and silicon nitride (Si₃N₄).

According to a second aspect of the present invention, there is provided a method for detecting a bioanalyte, the method comprising the steps of: contacting a sensing element of a sensor according to the first aspect with a sample solution comprising a bioanalyte; applying a voltage across the sensor; and detecting an electrical signal generated that is proportional to a change in conductance corresponding to a detection of the bioanalyte upon binding of the bioanalyte to the bioanalyte binding site.

Preferably, the bioanalyte binding site is a biomolecule.

In one embodiment, the bioanalyte binding site binds interleukin-6 (IL-6).

Suitably, the change in conductance detected in a sample solution with a concentration of IL-6 of 4 femtomolar is about 9.2%.

In one embodiment, the bioanalyte binding site binds C-reactive protein

Suitably, the change in conductance detected in a sample solution with a concentration of CRP of 13 femtomolar is about 12.5%.

According to a third aspect of the present invention, there is provided a method of fabricating a sensor for detecting a bioanalyte, the method comprising the steps of: providing a substrate; depositing a pair of terminal electrodes on the substrate in mutually spaced apart and opposing relation; and applying a non-insulating sensing element in the form of an oxygen-deficient metal oxide layer coated with a bioanalyte biding site, between and in electrical contact with the pair of terminal electrodes wherein the sensing element provides a conduction path between the terminal electrodes, wherein the bioanalyte binding site is selective toward detection of a bioanalyte upon binding of the bioanalyte to the bioanalyte binding site.

Preferably, the oxygen-deficient metal oxide layer is formed from a metal oxide selected from the group consisting of zinc oxide (ZnO), strontium titanium oxide (STO), tin oxide and titanium dioxide.

In one embodiment, the oxygen-deficient metal oxide layer has a thickness that falls within a range of about 50 nm to about 200 μm.

Preferably, the oxygen-deficient metal oxide layer is applied to the substrate surface by a technique selected from the group consisting of reactive sputtering, physical vapour deposition (PVD), chemical vapour deposition (CVD), metal organic chemical vapour deposition (MOCVD), pulsed laser deposition (PLD) and molecular beam epitaxy (MBE).

Suitably, the method further comprises the step of: physically or chemically adsorbing an intermediate layer to the oxygen-deficient metal oxide layer for anchoring the first biomolecule to the oxygen-deficient metal oxide layer.

Preferably, the intermediate layer is produced by silanization of the oxygen-deficient metal oxide layer with a silanizing agent having a terminal functionality that is selected from the group consisting of an epoxy group, a thiol group, an amino group, a carboxy group and a hydroxy group.

In one embodiment, the silanizing agent is selected from the group consisting of (3-glycidyloxypropyl)trimethoxysilane, (3-mercaptopropyl)trimethoxysilane (MTS), (3-aminopropyl)triethoxysilane (APTES), and N-(2-aminoethyl)-3-aminopropyl-trimethoxysilane (AEAPTS).

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a schematic representation for the fabrication of a non-invasive conductometric sensor for detecting a bioanalyte in accordance with a preferred embodiment of the present invention, in which the sensor has a sensing element that comprises an oxygen-deficient metal oxide thin film layer with a plurality of bioanalyte binding sites coupled thereto;

FIG. 2 shows plots that reflect the change in resistance (%) as a function of the concentration (M) of (A) IL-6 on an anti-IL-6 antibody immobilized conductometric sensor and (B) CRP on an anti-CRP antibody immobilized conductometric sensor. The dashed line in each plot indicates the antigen concentration (M) of healthy human body fluid (i.e. IL-6 in sweat and CRP in saliva);

FIG. 3 shows plots that reflect the change in resistance (%) of (A) CRP on an anti-IL-6 antibody immobilized conductometric sensor and (B) IL-6 on an anti-CRP antibody immobilized conductometric sensor, conducted for the purpose of a cross-selectivity study. The nominal concentrations (M) of IL-6 and CRP are 4 pM and 13 pM, respectively;

FIG. 4 shows core-level XPS spectra of (a,c) Zn 2p, (b,d) O 1s collected from sputtered ZnOₓ and ZnO_{y} thin films, respectively, formed on the substrate surface of the conductometric sensor of FIG. 1. Ov represents oxygen vacancies;

FIG. 5 shows resolved core-level XPS spectra of all three elements (a,d) Sr (b, e) Ti and (c, f) O in sputtered STO thin films with different chemical compositions formed on the substrate surface of the conductometric sensor of FIG. 1;

FIG. 6 shows plots that reflect the change in resistance (%) of (a) Selectivity for IL-6 antigens on IL-6 antibody immobilised devices in the presence of other antigens of study. (b) Selectivity for CRP antigens on CRP antibody immobilised devices in the presence of other antigens of study;

FIG. 7 shows plots that reflect (a) Change in resistance for fresh (Day 0) and old (Day 450) devices as a function of IL-6 concentration. (b) Change in resistance for fresh (Day 0) and old (Day 450) devices as a function of CRP concentration; and

FIG. 8 shows plots that reflect (a) Change in resistance as a function of IL-6 concentration in PBS and artificial saliva. (b) Selectivity study for IL-6 on IL-6 antibody immobilised-devices in the presence of other antigens used in the work. (c) Change in resistance as a function of CRP concentration in PBS and artificial saliva. (d) Selectivity study for CRP on CRP antibody immobilised-devices in the presence of other antigens used in the work.

### DETAILED DESCRIPTION OF SPECIFIC EMBODIMENTS

It should be noted in the following description that like or the same reference numerals in different embodiments denote the same or similar features.

The present invention is predicated on the finding of an inexpensive non-invasive sensor that employs a conductometric sensing technique for detecting the level of a range of bioanalytes in a bodily fluid such as human saliva and/or sweat for the prognosis/diagnosis of a medical condition. As will be described in more detail below, the conductometric sensor has a simple and comparatively easy-to-fabricate device structure, which offers a cost-effective alternative to conventional non-invasive sensors, that either require specialized substrates or adopt sensing techniques that limit their accuracy in results.

The inventors believe that the conductometric sensor as described in more detail below has compatibility with CMOS circuitry and could therefore be readily integrated with flexible/wearable electronics to provide a portable, personalized and reusable sensor that can be used to continuously monitor the levels of targeted bioanalytes through bodily fluids, without the need for invasive procedures. These bioanalytes can act as biomarkers indicative of the state and well-being of an individual.

What follows is a detailed description of the non-invasive conductometric sensor and a method for the application thereof for detecting the levels of a range of bioanalytes (such as biomarkers) in a bodily fluid.

Sensor

A sensor for use in detecting a bioanalyte according to a preferred embodiment of the present invention will now be described.

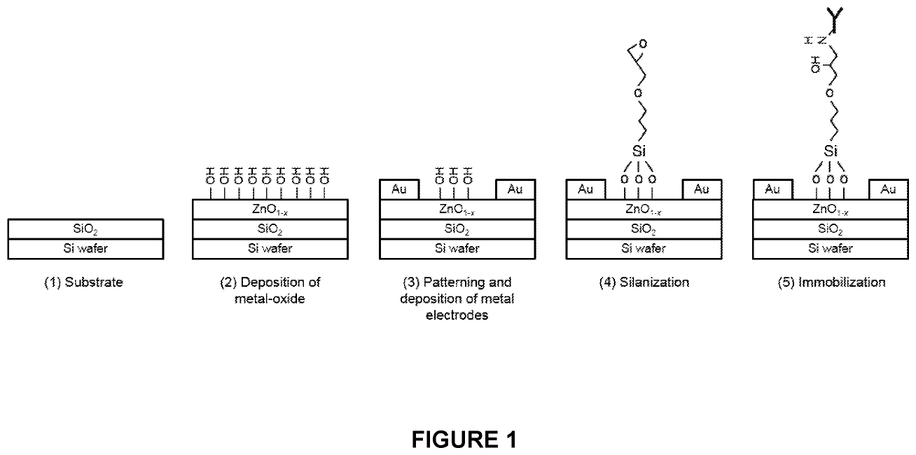

In its simplest form, and as shown in the schematic representation in FIG. 1, the sensor comprises a substrate, a pair of terminal electrodes disposed on the substrate in mutually spaced apart and opposing relation, and a non-insulating sensing element applied to a surface of the substrate, between and in electrical contact with the pair of terminal electrodes wherein the sensing element provides a conduction path between the terminal electrodes, and wherein the sensing element is comprised of an oxygen-deficient metal oxide layer that can be modified at the surface using suitable surface modification agents and synthetic binding entities or biomolecules to form sites capable of selectively binding a target biomarker or bioanalyte thereto for detection purposes.

What follows is a description of each of the components of the non-invasive conductometric sensor.

Substrate

The substrate may be manufactured from a material selected from the group consisting of a silicon wafer, a polymer, a glass or a ceramic.

For instance, suitable polymers for use as the substrate may be selected from the group consisting of polydimethylsiloxane (PDMS), polyimide (PI) and polyethylene naphthalate (PEN). While suitable ceramics may be selected from the group consisting of aluminium oxide (Al₂O₃), sapphire and silicon nitride (Si₃N₄).

Here, for the purpose of describing the steps for manufacturing a non-invasive conductometric sensor, and as shown in step (1) of FIG. 1, the substrate is a rigid silicon wafer having a SiO₂ surface.

It will be appreciated by persons of ordinary skill in the relevant art however, that if the desired purpose is to provide a non-invasive conductometric sensor that can be used as a device in applications where portability and flexibility is desirable, then the substrate used is ideally a flexible polymer such as a polyimide foil rather than the rigid SiO₂/Si wafer described above. The steps for manufacturing a flexible non-invasive conductometric sensor using the polyimide foil are the same as described above (see FIG. 1).

Sensing Element

In its simplest form, the sensing element comprises an oxygen-deficient metal oxide layer and one or more bioanalyte binding sites bound to the surface of the oxygen-deficient metal oxide layer by either chemical or physical adsorption.

The metal oxide layer may be formed using any suitable metal oxide selected from the group consisting of zinc oxide (ZnO), strontium titanium oxide (STO), tin oxide and titanium dioxide.

In a preferred form, the metal oxide layer is an oxygen-deficient metal oxide layer formed using zinc oxide (ZnO) or strontium titanium oxide (STO). As will be described below, the inventors have found that good results may be obtained when the metal oxide layer is a thin film oxygen-deficient zinc oxide (ZnO) layer.

The oxygen-deficient metal oxide layer may be applied to the substrate surface by a technique selected from the group consisting of reactive sputtering, physical vapour deposition (PVD), chemical vapour deposition (CVD), metal organic chemical vapour deposition (MOCVD), pulsed laser deposition (PLD) and molecular beam epitaxy (MBE).

In a preferred embodiment, and as shown in step (2) of FIG. 1, the oxygen-deficient metal oxide layer is applied to the surface of a rigid (SiO₂/Si) wafer or a flexible polyimide foil by reactive sputtering to afford a thin metal oxide film having a thickness that falls within a range of about 50 nm to about 200 μm.

For instance, as shown in step (2) of FIG. 1, zinc oxide has been sputtered onto the surface of a rigid (SiO₂/Si) wafer to provide an oxygen-deficient zinc oxide layer (ZnO₁₋ₓ) that presents a plurality of hydroxy (OH) groups at the surface. The as-deposited oxygen-deficient ZnO layer may be of any suitable thickness to suit the desired application.

Good results have been obtained when the oxygen-deficient ZnO layer has a thickness that falls within the range of about 10 nm to about 1 μm.

Metal Oxide Thin Films

Different binary (ZnOₓ and ZnO_{y}) and complex metal oxide (such as SrTiO₃) thin films have been engineered for the application of non-invasive conductometric sensors for sensing different bioanalytes such as, for example, IL-6 and CRP.

The following section discusses the synthesis process and chemical composition of these binary and complex metal oxide thin films.

Zinc Oxide (ZnO)

Two different types of ZnO thin films with different ratios of oxygen content are prepared via magnetron sputtering. This results in different stoichiometries of the sputtered thin films. The sputtering parameters and associated conductivity are listed in Table 1.

TABLE 1
Parameters used to sputter ZnO thin films with different
stoichiometries and associated electrical conductivity.

| | | | | Processing | Processing | |
|---|---|---|---|---|---|---|
| Thin Film | Sputtering | Power | | pressure | Temperature | Conductivity |
| Types | Source | (W) | Ar:O₂ ratio | (mTorr) | (° C.) | (S/m) |
| ZnOₓ | Zn (metallic) | 200 | 100:30 | 3.5 | 250 | 0.08-0.6 |
| ZnO_{y} | ZnO | 200 | 100:05 | 3.5 | 250 | <0.07 |
| | (ceramic) | | | | | |

The sputtering parameters are selected to engineer thin films with electrical conductivities in the range of 0.08-0.6 S/m. This range of conductivity gives maximum sensitivity of the sensors.

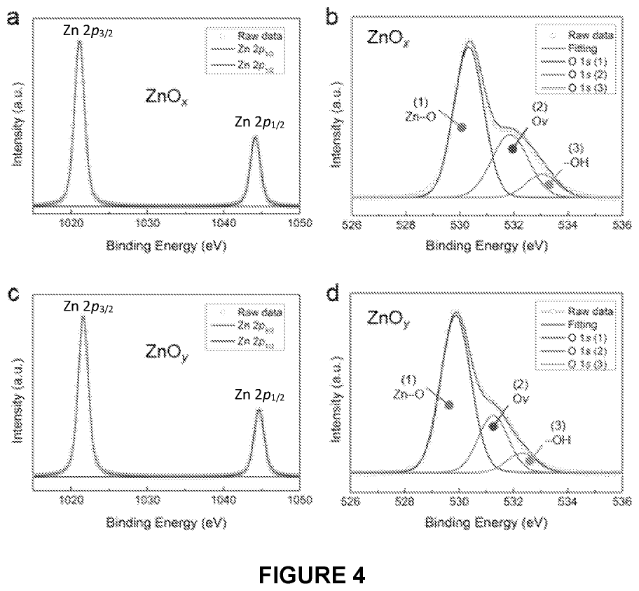

The stoichiometry of the sputtered ZnO thin films is assessed by the X-ray photoelectron spectroscopy (XPS). FIG. 4 shows the core-level Zn 2p and O 1s spectra collected from ZnOₓ and ZnO_{y} thin films.

As shown in FIG. 4, the O 1s spectra are fitted with three distinct peaks associated with Zn—O bonding (represented as peak (1)), oxygen vacancies (Ov, represented by (2)) and —OH bonding (marked as (3)).^{[1,2]}

The fitting parameters are listed in Table 2. The relative comparison of peaks(2) in both types of thin films suggest that ZnO is relatively more oxygen deficient than ZnO_{y}.

TABLE 2
Fitting parameters of core-level XPS spectra
collected from ZnOₓ and ZnO_{y} thin films.

| | Peak position (eV) | | | | | | | |
|---|---|---|---|---|---|---|---|---|
| | | | O 1s | | | | | |
| Thin Film | | | | (1) | | (2) | | (3) |
| Types | Zn 2p_{3/2} | Zn 2p_{1/2} | (1) | At. % | (2) | At. % | (3) | At. % |
| ZnOₓ | 1021.60 | 1044.66 | 530.32 | 59.57 | 531.85 | 30.66 | 533.03 | 9.76 |
| ZnO_{y} | 1021.10 | 1044.16 | 529.88 | 67.15 | 531.25 | 24.48 | 532.30 | 8.36 |

Strontium Titanium Oxide (STO)

Two different types of strontium titanium oxide (SrTiO₃: STO) thin films are prepared via magnetron sputtering with different ratios of oxygen content. The sputtering parameters are summarised in Table 3.

TABLE 3
Parameters used to sputter STO thin films with different
stoichiometries and associated conductivity.

| | | | | Processing | Processing | |
|---|---|---|---|---|---|---|
| Thin Film | Sputtering | Power | | pressure | Temperature | Conductivity |
| Types | Source | (W) | Ar:O₂ ratio | (mTorr) | (° C.) | (S/m) |
| STOₓ | Ceramic | 200 | 100:00 | 3.5 | 23 | 0.07-0.09 |
| | SrTiO₃ | | | | | |
| STO_{y} | Ceramic | 200 | 100:05 | 3.5 | 23 | <0.04 |
| | SrTiO₃ | | | | | |

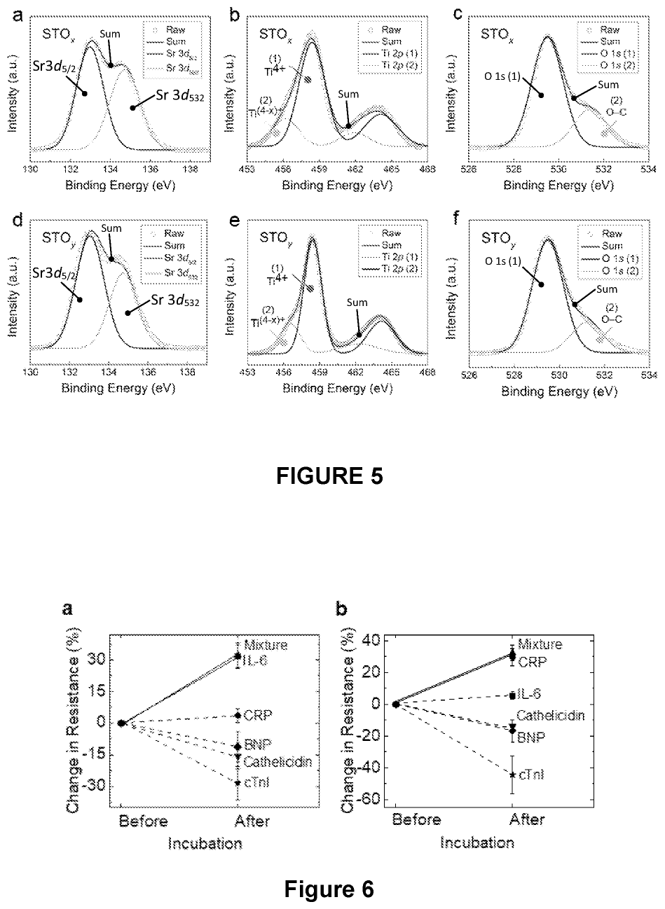

FIG. 5 shows the XPS core-level binding energy spectra of all three elements in both types of STO thin films, namely STOₓ and STO_{y} sputtered in a reducing (0% oxygen) and an oxidizing (5% oxygen) environment, respectively. In both STO oxides, the core-level spectra of Sr 3d (FIGS. 5a, 5d) is fitted by a single component with no significant shift observed in chemical states. The binding energies for Sr 3d_{5/2} at 132.9 eV (±0.1 eV) and for Sr 3d_{3/2} at 134.7 eV (±0.1 eV) for both oxides, are attributed to Sr²⁺ species in STO.^{[3, 4]}

The analyses of the de-convoluted spectra of the core level binding energies of Ti 2p are shown in FIGS. 5b, 5e. Both Ti 2p spectra, in STOₓ and STO_{y}, spectra are fitted with two distinct components, i.e., Ti⁴⁺ and Ti^{(4-x)+}. The presence of solely a Ti⁴⁺ component corresponds to a fully stoichiometric STO oxide, while the Ti^{(4-x)+} species (such as Ti³⁺ and Ti²⁺) represent the presence of oxygen vacancies in the oxide system. However, Ti³⁺ and Ti²⁺ components are fitted with only one component at the lower binding energies and denote as Ti^{(4-x)+} to avoid any ambiguity. In STOₓ and STO_{y} oxides, the peaks at Ti 2p_{3/2} binding energies of 458.4 eV are assigned to Ti⁴⁺ oxidation state while the peaks at 456.2 eV and 456.5 eV are assigned to Ti^{(4-x)+} species.^{[5-8]} The relative ratio of the individual Ti⁴⁺ and Ti^{(4-x)+} species are calculated by integrating the fitted peaks. The relative ratios of Ti⁴⁺ and Ti^{(4-x)+} in STOₓ are calculated to be 72.9% and 27.1%, respectively.

On the other hand, the relative ratios of Ti⁴⁺ and Ti^{(4-x)+} species in STO_{y} are calculated to be 75.2% and 24.8%, respectively. This indicates that the concentration of Ti^{(4-x)+} species in STOₓ is comparatively higher than in the STO_{y}. As such, STOₓ thin films are more oxygen deficient than STO_{y} thin films.

Furthermore, O 1s spectra (FIGS. 5c, 5f) are fitted with two components with peak positions at 529.5 eV and 531.3 eV, corresponding to O²⁻ ions in STO oxide^{3]} and C—O bond^{[3, 4, 9]} formed due to adsorption of adventitious carbon onto the surface, respectively.

Electrodes

As shown in step (3) of FIG. 1, a pair of gold terminal electrodes is formed on the surface of the as-deposited oxygen-deficient metal oxide thin film in mutually spaced apart and opposing relation, and in electrical contact with the oxygen-deficient metal oxide thin film acting as the sensing element of the sensor.

Briefly, the terminal electrodes are formed by evaporating a gold thin film (250 nm with 100 nm chromium adhesion layer) on top of the oxygen-deficient metal oxide layer using electron beam lithography. The as deposited gold thin film is then patterned using standard photolithography and wet etching techniques to define the pair of terminal electrodes.

Intermediate Layer

In a preferred form, the bioanalyte binding sites are anchored to the oxygen-deficient metal oxide layer via an intermediate layer formed using a plurality of long chain molecules that have been either physically or chemically adsorbed to the oxygen-deficient metal oxide layer as surface modification agents.

In one embodiment, the intermediate layer is produced by silanization of the hydroxy groups of oxygen-deficient zinc oxide layer with a silanizing agent having a terminal functionality that is selected from the group consisting of an epoxy group, a thiol group, an amino group, a carboxy group and a hydroxy group.

For instance, as shown in step (4) of FIG. 1, the silanizing agent is the epoxy-terminated silanizing agent, (3-glycidyloxypropyl)trimethoxysilane (GPS). The protocol for adsorbing GPS to the oxygen-deficient zinc oxide layer is described in the Materials and Methods section below.

In other embodiments, the surface modification agent may be selected from the group consisting of (3-mercaptopropyl)trimethoxysilane (MTS), (3-aminopropyl)triethoxysilane (APTES), and N-(2-aminoethyl)-3-aminopropyl-trimethoxysilane (AEAPTS).

Analyte Binding Site

As shown in step (5) of FIG. 1, the now silanized surface of the oxygen-deficient zinc oxide layer is further modified by immobilizing a suitable binding entity or biomolecule to the terminal end of each of the anchored silanizing agents to act as a binding site for the selective binding of a desired bioanalyte from a biological sample.

There is a range of biomolecules that may be utilised as binding sites for the for the selective binding of a desired bioanalyte from a biological sample.

For instance, such biomolecules may include proteins, peptides, lipo-peptides, protein-binding carbohydrates or protein-binding ligands.

In one embodiment, the biomolecule is a capture protein.

Suitably, the capture protein is a protein-binding scaffold, a T-cell receptor, a binding-fragment of a TCR, a variable lymphocyte receptor, an antibody and/or a binding-fragment of an antibody.

Protein Binding Scaffolds

Protein binding scaffolds have emerged as viable molecules for binding with a diverse range of bioanalytes including proteins. Protein binding scaffolds typically comprise a stable protein structure (scaffold) which can tolerate modification of amino acids within designated binding regions without alteration of the relative arrangement of the binding domains. These protein-binding scaffolds include (but are not limited to): Adnectins, Affilins (Nanofitins), Affibodies, Affimer molecules, Affitins, Alphabodies, Aptamers, Anticalins, Armadillo repeat protein-based scaffolds, Avimers, Designed Ankyrin Repeat Proteins (DARPins), Fynomers, Inhibitor Cystine Knot (ICK) scaffolds, Kunitz Domain peptides, Monobodies (AdNectins™) and Nanofitins.

Affilins are artificially created proteins of about 20 kDa. They include scaffolds that are structurally related to human ubiquitin and vertebrate gamma-B crystallin, with eight surface-exposed manipulatable amino acids. Affilins can be designed to bind specifically to target bioanalytes and can be specifically adapted to biding to a large variety of molecules using techniques such as site-directed mutagenesis and phage display libraries.^{[10]}

Affibodies are proteins of about 6 kDa which comprise the protein scaffold of the Z domain of the IgG isotype antibody with modification to one or more of 13 amino acid residues located in the binding domains of its two alpha-helices.^{[11]}

Affimer molecules are proteins of about 12 to 14 kDa which utilise a protein scaffold derived from the cysteine protease inhibitor family of cystatins. Affimer molecules contain two peptide loop regions in addition to an N-terminal sequence which can be adapted for target-specific binding. Affimer molecules, having 1010 combinations of amino acids at the binding sites, can be generated using phage display libraries and appropriate techniques.^{[12]}

Affitins are proteins of 66 amino acid residues (about 7 kDa) and use a protein scaffold derived from the DNA binding protein Sac7d found in Sulfolobus acidocaldarius. They are readily produced in vitro from prokaryotic cell cultures and contain 14 binding amino acid residues which can be mutated to produce in excess of 3×10¹² structural variants.^{[13]} Screening techniques such as surface plasmon resonance can be used to identify specific binding of these molecules.

Alphabodies are approximately 10 kDa molecules that, unlike most macromolecules, can penetrate the cellular membrane (when not immobilised) and therefore can bind to intracellular and extracellular molecules. The scaffold of Alphabodies are based on computationally designed coiled-coil structures with three alpha-helices (A, B and C) which are not analogous to natural structures. Amino acids on the A and C alpha-helices can be modified to target specific antigens.^{[14]}

Aptamers for binding to proteins include a range of nucleic acids (DNA, RNA and XNA) and peptides, which can be screened for binding to specific target molecules. Databases of nucleic acid aptamers ^{[15]} allow for the selection of in vitro identified DNA aptamers. Peptide aptamers consist of short amino acid sequences that generally are embedded in a looped structure within a stable protein scaffold frame (a “loop on a frame”). Typically, a 5 to 20 residue peptide loop is the source of variability for selective biding to target molecules. Combinatorial libraries and techniques such as yeast-two hybrid screening can be used to generate and screen peptide aptamers. Other techniques for generating and screening of protein aptamers are described in the literature.^{[16]}

Anticalin proteins are protein binding molecules that are derived from lipocalins. Typically, anticalins bind to smaller molecules than antibodies. Methods for screening and developing anticalins are described in the literature.^{[17, 18]}

Armadillo repeat protein-based scaffolds are characterized by an armadillo domain, composed of tandem armadillo repeats of approximately 42 amino acids, formed into a super-helix of repeating units composed of three α-helices each. Modification of residues within the conserved binding domain allow for preparation of a range of combinatorial libraries which can be used for selection of target-specific binders.^{[19]}

Avimers (also known as avidity multimers, maxibodies or low-density lipoprotein receptor (LDLR) domain A) comprise at least two linked 30 to 35 amino acid long peptides based on the A domain of range of cysteine-rich cell surface receptor proteins. Modification of the A domain allows for directed binding to a range of epitopes on the same target or across targets, with the number of linked peptides determining the number of possible targets per avimer. A range of avimer phage display libraries are known in the art including commercial libraries such as those of Creative Biolabs.

Designed Ankyrin Repeat Proteins (DARPins) are engineered binding proteins derived from ankyrin proteins. Methods for screening and identifying DARP ins are described in the literature. ^{[20, 21]}

Inhibitor Cystine Knot (ICK) scaffolds are a family of miniproteins (30 to 50 amino acid residues long) which form stable three-dimensional structures comprising three disulphide bridges connecting a series of loops having high sequence variability. Inhibitor Cystine Knots include three family members being knottins; cyclotides and growth factor cysteine-knots. Databases are known in the art, such as the KNOTTIN database (www.dsimb.inserm.fr/KNOTTIN/) which disclose specific properties of known Knottins and cyclotides, such as their sequence, structure and function. Further, methods for producing ICKs and screening for binding are described in the literature.^{[22]}

Monobodies (also known under the trade name AdNectins) utilise an FN3 (fibronectiv type III domain) scaffold with diverse and manipulatable variable groups. Adnectis share antibody variable domains and a beta-sheet loop with antibodies. The binding affinity of monobodies can be diversified and customised by in vitro evolution methods such as mRNA display, phage display and yeast display. Methods for screening and producing monobodies are described in the literature.^{[23, 24]}

Antibodies and Antibody Fragments

In some embodiments, the bioanalyte binding site is an antibody, or a binding fragment thereof. Antibodies are protein binding molecules that have exemplary diversity with potentially as many as 10¹¹ to 10¹² unique molecules in a single individual, with genetic variation between individuals allowing for further diversity. Antibody diversity in vivo is driven by random recombination of a series of genes in V(D)J joining.

The binding of an antibody is determined primarily by the three hypervariable regions of the heavy and light chain, termed complementarity-determining regions (CDR) 1, 2 and 3. As such, each mature antibody has six CDRs (variable heavy (VH) chain CDR1, CDR2 and CDR3 and variable light (VL) chain CDR1, CR2 and CDR3). These hypervariable regions form the three-dimensional antigen-binding pocket, with the binding specificity of the antibody determined by the specific amino acid sequences in the CDRs, primarily CDR3.

Antibodies to specific bioanalytes may be obtained commercially or generated by methods known in the art. For example, antibodies to specific bioanalytes may be prepared using methods generally disclosed in the literature.^{[25]}

The specificity, avidity and affinity of antibodies generated within subjects can be modified by way of in vitro processes such as affinity maturation.^{[26]} As such, in vivo derived antibodies can be further modified to produce distinct, yet lineally related, antibodies. Consequently, the term “antibody” encompasses in vivo derived antibodies and in vitro derived molecules that have undergone processes of mutation to modify the CDR binding sites, such that they have unique sequences when compared to the antibodies generated in vivo.

The term antibody also includes non-conventional antibodies generated from species such as camelids, shark and jawfish. As such, the term antibody includes heavy-chain antibodies including camelid antibodies, IgNARs and variable lymphocyte receptors (VLRs). Further, these can be fragmented into their biding portions (such as VNARs—single binding portion of IgNARs) or integrated recombinantly into a fusion protein. Methods are described in the literature for generating and adapting such non-conventional antibodies. ^{[27, 28]}

Antibody Binding Fragments

In some embodiments, the bioanalyte binding site is an antibody binding fragment. Antibody binding fragments can be derived from an antibody or may be recombinantly generated with sequences identical to the CDRs of an antibody or antibody fragment. Indeed, these CDRs may be from an affinity matured antibody and therefore may not be identical to an in vivo derived antibody.

Antibodies are comprised of four chains (two heavy and two light chains) and can be separated into the Fc (fraction crystallisable) and the Fab (fraction antibody) domains. The Fc portion of the antibody interacts with the Fc receptor and the complement system. Consequently, the Fc portion is important for the immune function of the antibody. However, the Fab portion contains the binding regions of the antibody and is critical for the specificity of an antibody for the desired epitope.

Accordingly, in some embodiments, the bioanalyte binding site is a Fab fragment of an antibody. Fab fragments can be individual Fab fragments (i.e. the antibody fragment is generated in the absence of linking disulphide bridges) or an F(ab′)2 fragment which comprises the two Fab fragments of an antibody linked via disulphide bridges. These fragments are typically generated by fragmenting an antibody using digestion enzymes, such as pepsin. Methods are described in the literature. ^{[29]}

Each Fab fragment of an antibody has six CDRs in total with the VH and VL chains comprising three CDRs each (within a framework consisting of four framework regions). The constant regions of the Fab fragment can be removed to leave only the VH and VL regions of an antibody. Individual VH and VL chains (each only comprising three CDRs) have been shown to bind specifically with high affinity. Typically, individual binding regions are known as single antibody domains (sdAbs). Alternatively, the VH and VL chains can be linked via a linker to form a fusion protein known as a single-chain variable fragment (scFv—also known as a diabody). Unlike Fabs, scFvs are not fragmented from an antibody, but rather are typically recombinantly formed based on the CDR and framework regions of an antibody. Further, sdAbs can also be recombinantly produced and form the binding component of a larger fusion protein which may also include a portion that may act to stabilise the binding region, improve or facilitate anchoring to the sensing element or the intermediate layer, improve binding by, for example, providing flexibility of the biding region or optimizing the length of the bioanalyte binding site thereby allowing access to the antigenic region of the bioanalyte. Consequently, in some embodiments, the bioanalyte binding site is, or includes, a scFv or an sdAb. The scFv may include multiple VH and VL chains linked together to form a multivalent scFv, such as a di-scFv or a tri-scFv.

Antibodies to specific bioanalytes and fragments of antibodies, or fusion proteins containing antibody derived sequences may be obtained commercially or generated by methods known in the art, such as those discussed above.

Proteins and Receptors

Protein receptors or ligands which interact with and bind to proteins may be used as a bioanalyte binding site. Such receptors and ligands include whole receptors or ligands, or specific fragments thereof (e.g. a fragment comprising a binding domain of the receptor or ligand). Specifically-envisaged receptors include receptors for cytokines, such as interleukins, or chemokines which can be informative on the state of the immune system. In some embodiments, the receptor or ligand (or fragment thereof) may be integrated to form a fusion protein.

For instance, interleukin-6 (IL-6) is an inflammatory pluripotent cytokine and is an important biomarker that can be used to monitor the immune response during cancer treatment. It can also be used to monitor psychological stress and insulin activity.

For instance, the inventors have obtained good results when using anti-interleukin-6 (IL-6) antibodies for the selective recognition and binding of IL-6.

For instance, the inventors have obtained good results when using anti-C-reactive protein (CRP) antibodies for the selective recognition and binding of CRP.

### SUMMARY

In short, the non-invasive conductometric sensor described above is a passive electronic device configured with a simple in-plane two-terminal electrode geometry, in which a sensing element of the sensor takes the form of an oxygen-deficient metal oxide thin film that has been applied to the surface of the sensor substrate and subsequently functionalized with specific bioanalyte binding sites that are selective towards one or more bioanalytes to be detected in a bodily fluid such as human saliva and/or sweat. When a voltage is applied across the sensor, an electrical signal is generated that is proportional to a change in conductance of the sensing element that is produced as a result of charge transfer between the complex formed between the bioanalytes and the bioanalyte binding sites and the oxygen-deficient metal oxide thin film layer. This electrical signal can be equated to the level of the target biomarker or bioanalyte present within the bodily fluid.

What now follows is a description of the method for detecting the level of a target bioanalyte in a bodily fluid using the non-invasive conductometric sensor described above.

Detection Method

According to another preferred embodiment of the present invention, there is provided a method for detecting a bioanalyte,

Briefly, the method for detecting a bioanalyte using the oxygen-deficient metal oxide based sensor comprises the following steps of: (i) contacting the oxygen-deficient metal oxide-based sensing element with a sample solution of a bodily fluid comprising a bioanalyte; (ii) applying a voltage across the sensor; and (iii) using a current source meter to detect the electrical signal generated between the pair of terminal electrodes that is proportional to a change in conductance corresponding to a detection of the bioanalyte upon binding of said bioanalyte to the bioanalyte binding sites on the surface of the oxygen-deficient metal oxide sensing element.

### EXAMPLES

Antigen Concentration Dependent Study:

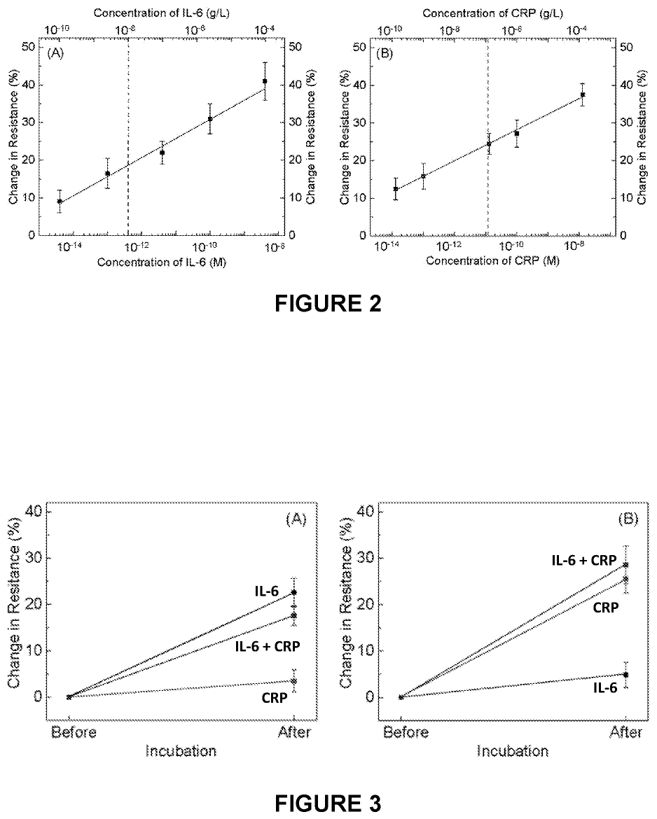

Both IL-6 and CRP antigens show a concentration dependent resistance change with respect to the baseline resistance of the devices. The baseline resistance was measured for the antibody immobilized-GPS silanized sensors prior the addition of antigen. A linear correlation is observed for the change in resistance, as a function of antigen concentration, for both IL-6 and CRP antigens (FIG. 2). The responsivity (i.e. the slope of the curve) for IL-6 and CRP are 5.1 and 4.1%/M, respectively. These values suggest that the anti-IL-6 antibody-immobilized ZnO sensors exhibit a greater sensitivity toward detecting IL-6 antigens compared to the anti-CRP antibody-immobilized ZnO conductometric sensors in detecting CRP antigens. To determine the contribution from the matrix of the antigen solution, the resistance was measured for PBS solution on both types of antibody-immobilized ZnO sensors. The change in resistance in the presence of PBS solution for both types of antibody-immobilized sensors is less than 1%. As such, the contribution from the matrix of the antigen solution for the change in resistance is negligible.

For both antigens, the non-invasive conductometric ZnO sensor demonstrated a detectable response for the concentrations even below the healthy human body fluid. The reported IL-6 concentration in healthy human sweat is around 0.38 pM (10 ng/L)^{[30]} and the CRP concentration in healthy human saliva is around 12 pM (285 ng/L).^{[31]} The ZnO conductometric sensors show a 9.2% change of resistance for the IL-6 concentration of 4 fM which is more than 100 times lower in IL-6 concentration of healthy human sweat. Similarly, the change of resistance for the lowest CRP concentration detected by the sensor (13 fM) is ˜12.5% which is almost 1000 times lower in CRP concentration of healthy human saliva. This high sensitivity of the responses for the concentrations far below healthy human body fluids clearly indicate the importance of ZnO-based conductometric sensors in detecting bioanalytes in human body fluids.

Cross-Selectivity Study 1:

To determine the viability of each antibody-immobilized device in the presence of other antigens, a cross-selectivity study was conducted. Anti-IL-6 antibody immobilized devices demonstrated 3% change in resistance in the presence of 13 pM CRP while CRP antibody immobilized devices demonstrated 3.5% change in resistance in the presence of 4 pM IL-6 (FIG. 3). The antigen concentrations used in this experiment were selected as close as possible to the antigen concentration of healthy human body fluids. When both antigens are mixed prior the addition onto the devices, anti-IL-6 antibody immobilized devices displayed 17.6% change in resistance which is ˜4% lesser compared to the 4 pM IL-6 on the same device. In contrast, the change in resistance for anti-CRP antibody immobilized devices is 27.4% which is ˜3% higher compared to the 13 pM CRP on the same device. The cross-selectivity test results clearly indicate that anti-IL-6 antibody immobilized ZnO devices are selective to IL-6 in the presence of CRP antigens and anti-CRP antibody immobilized ZnO devices are selective to CRP antigens in the presence of IL-6 antigens. The reason for the decreased 4% change on IL-6 antibody devices and increased 3% change on CRP antibody devices in the presence of antigen mixture is currently unclear.

Cross-Selectivity Study 2:

To determine the viability of each antibody-immobilized device in the presence of other antigens, a cross-selectivity study was conducted. Cathelicidin, B-natriuretic peptide (BNP) and cardiac troponin I (cTnI) were used along with IL-6 and CRP antigens for this study. The nominal concentrations of the pristine antigen and antigens in the mixture were maintained at 4 pM. A significantly high resistance change was observed for the targeted antigen when the antigen mixture was reacted with the corresponding antibody-immobilised devices (FIG. 6). 31% resistance change was observed for IL-6 in the antigen mixture when treated with the IL-6 antibody-immobilised devices. Only 3% resistance change was observed for CRP. Furthermore, CRP antibody-immobilised devices displayed 30% resistance change for CRP in the antigen mixture while IL-6 contributed 5% resistance change. It is important to note that there were no contributions from cathelicidin, BNP, and cTnI to the resistance changes in both IL-6 and CRP antibody-immobilised devices. The small contributions from IL-6 and CRP antigens to the resistance changes in their non-corresponding antibodies could possibly due to the physical adsorption of these molecules onto the antibodies. The negative contribution in resistance changes in cathelicidin, BNP, and cTnI suggests that either these 3 antigens have not bounded to the antibodies studied or the charge transfer effect of these antigens adopts a different mechanism than CRP and IL-6 antigens on the antibodies.

Materials and Methods

Fabrication of Sensors:

The sensors were fabricated on rigid (SiO₂/Si) and flexible plastic (polyimide foils) substrates by depositing a 100 nm thick thin film of a metal oxide, such as zinc oxide (ZnO) acting as a sensing element in the sensors. The composition of the sensing element is engineered by reactive sputtering to produce an oxygen deficient metal oxide film with a conductance in the range of 0.08-2 siemens/m², more preferably in the range of 0.08-0.6 siemens/m². For conductance measurements, two terminal in-plane electrodes are patterned with a sensing area of 16×10⁻⁶ m². The change in conductance corresponding to dispensed antibody and antigen is measured using a commercial current source meter (B2901A precision source/measure unit from Keysight Technologies).

Preparation of Antibody and Antigen Solutions:

Interleukin-6 (IL-6), anti-IL-6, C reactive protein (CRP) and anti-CRP were purchased from a commercial vender (Sigma-Aldrich) and used as-received. Cathelicidin, B-natriuretic peptide (BNP) and cardiac troponin I (cTnI) were sourced from multiple commercial vendors (Abcam, MyBioSource, and ProSpec Bio) and used as-received.

The anti-IL-6 stock solution was diluted by 1:10⁶ in phosphate buffer solution (PBS, pH 7.4) to immobilize on the surface of the oxygen-deficient ZnO sensors. As-received anti-CRP solution was diluted by 1:50 in PBS (pH 7.4) prior to the immobilization. As-received IL-6 powder was completely dissolved in a known amount of autoclaved Milli-Q water and diluted in pH 7.4 PBS solution to prepare a standard series of IL-6 solutions. The IL-6 concentrations prepared are 4 nM, 100 pM, 4 pM, 100 fM, and 4 fM. A standard series of CRP solutions was also prepared by diluting the as-received CRP solution in pH 7.4 PBS with predetermined volumes. The prepared CRP concentrations are 13 nM, 100 pM, 13 pM, 100 fM, and 13 fM. Solutions of the other antigens were prepared in a similar way.

Preparation of GPS-Silanized Sensors:

Silanization of oxygen-deficient metal oxide sensor surfaces using (3-glycidyloxypropyl)trimethoxy silane (GPS) has been reported before, albeit for invasive sensors.^{[32]} Herein, the silanization of ZnO was conducted with few modifications to the reported silanization procedure. In brief, freshly prepared ZnO devices were exposed to O₂ plasma for 10 minutes (Plasma Cleaner PDC-002, Harrick Plasma) to activate the hydroxyl groups on the ZnO surface. Then, 20 μL of freshly prepared GPS solution was drop-casted onto an Al foil, which was placed inside a vacuum desiccator, allowing GPS vapor to build up inside the desiccator. Then, the O₂ plasma cleaned-ZnO sensors were exposed to this GPS vapor for 30-45 min. The exposure of the plasma cleaned ZnO sensors to GPS vapor was conducted inside an LC 200 Glovebox System. Upon completion of the exposure to GPS vapor, the ZnO sensors were rinsed thoroughly with Milli-Q water for 2 minutes to remove any unbound silane groups from the surface of the ZnO sensors. Then, the washed ZnO sensors were heated at 150° C. for 10 minutes to strengthen the bonding of the silane groups to the ZnO surface. These GPS-silanized sensors were then used for the immobilization of antibodies.

It will be appreciated that the silanization of an oxygen-deficient strontium titanium oxide (STO) sensor surface using (3-glycidyloxypropyl)trimethoxy silane (GPS) or an alternative silanizing agent can be carried out in the same manner as described above.

Immobilization of Antibodies:

Antibody (IgG) immobilization on GPS-silanized ZnO sensors has been reported before, albeit for invasive sensors.^{[32]} A 25 μL volume of freshly prepared 1:10⁶ dilution anti-IL-6 solution was drop casted on to the surface of each of the freshly GPS-silanized ZnO sensors and incubated for 2 hours allowing the IL-6 antibodies to immobilize on the surface of the ZnO sensors. Then, the sensors were rinsed with pH 7.4 PBS solution to remove any unbounded antibodies. The PBS-washed ZnO sensors were then dried in a stream of N₂ gas. These anti-IL-6 antibody immobilized sensors were used for the IL-6 antigen concentration and cross-selectivity measurements. The same procedure was followed to prepare the CRP-immobilized GPS-silanized ZnO sensors by using 25 μL of freshly prepared 1:50 diluted anti-CRP solution.

Conductometry of Antigens

The baseline conductance of the antibody-immobilized ZnO sensors was measured prior to the addition of the antigen. A 15 μL volume of the antigen solution was drop casted on the surface of the antibody-immobilized ZnO sensors and incubated for 10 minutes. After such time, the remaining antigen solution on the sensor was removed and the surface was dried under a stream of N₂ gas. Followed by conductance measurements of each concentration. For the cross-selective measurements, 4 pM of IL-6 solution (15 μL) was drop casted on anti-CRP antibody immobilized ZnO devices and 13 pM CRP solution (15 μL) was drop casted on anti-IL-6 antibody immobilized ZnO devices. The nominal concentrations of IL-6 and CRP antigens in the pre-mixed solution for cross-selectivity measurements were 4 pM and 13 pM, respectively.

Device Shelf-Life Study

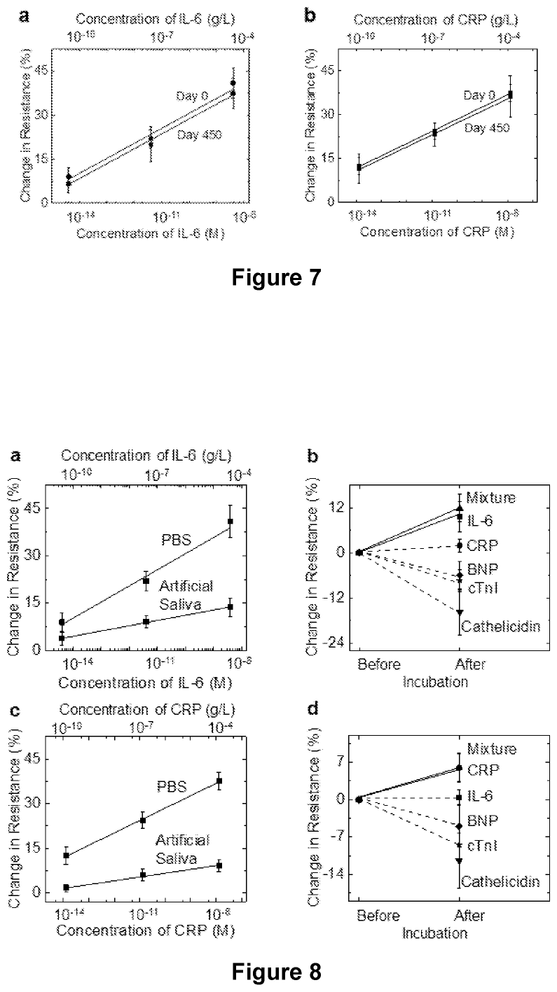

The GPS-silanised devices are stable for at least 15 months. This conclusion was derived based on the resistance changes of IL-6 and CRP antigens obtained for 15 months (450 days) old devices (FIG. 7). The change in resistance for both types of antigens displayed a linear variation with their concentrations. A similar linear trend was observed for freshly prepared devices (days 0) for both types of antigens. The close overlap of the standard errors of the resistance changes for a given concentration of the selected antigen strongly suggests there is no statistical difference between average resistance change values of fresh and old devices for a given concentration of a selected antigen.

Device Performances in Artificial Saliva

The IL-6 and CRP antibody immobilised-devices were successfully detected the corresponding antigens in artificial saliva (FIG. 8). The change in resistance for both types of antigens was linearly varied with their concentration. The responsivity (i.e. slope) for IL-6 and CRP in artificial saliva are 1.6 (%/M) and 1.2 (%/M), respectively suggesting the devices are more sensitive to IL-6 than CRP in artificial saliva. The resistance change for a given concentration of these antigens is significantly low in artificial saliva with respect to the resistance change in PBS. The large background contribution from artificial saliva (83% for IL-6 and 85% for CRP) has caused to this significant lower resistance changes. This could possibly due to the high ionic composition of artificial saliva compared to the PBS causing the higher charge transfer effect. With similar to the effect in PBS, these devices displayed a significant selectivity for the targeted antigen in artificial saliva when the devices are immobilised with corresponding antibodies. As an example, the resistance change for the IL-6 in the antigen mixture prepared in artificial saliva is significantly higher than the other antigens in the presence of IL-6 antibody-immobilised devices. Similarly, CRP antibody immobilised devices displayed a significantly high resistance change for CRP antigens in antigen mixture prepared in artificial saliva.

Advantages

From the foregoing discussion, it should be apparent to a skilled person in the art that the non-invasive oxygen-deficient metal oxide-based conductometric sensors of the various embodiments of the present invention provide many advantages over their existing counterparts.

Indeed, the oxygen-deficient metal oxide-based conductometric sensors are capable of measuring concentrations of targeted bioanalytes that are lower than their respective levels in human bodily fluids. To this end, the inventors believe that such sensors offer significant potential for the development of cost-effective, bio-compatible and functional sensors that may find wide application as personalised and reusable healthcare monitoring devices. In fact, it is widely anticipated by the inventors that these sensors may have significant impact on the following:
1. Cardiovascular disease warnings: Routine testing with these sensors are anticipated to warn of elevated levels of inflammatory biomarkers to intervene in advance of a heart attack or stroke;
2. Cancer treatment: These sensors are anticipated to be invaluable in monitoring the levels of biomarkers for cancer and its treatment; and
3. Abdominal disorder treatment: These sensors are also anticipated to sense abdominal disorders such as live disorders via sensing CRP antigens.

The oxygen-deficient metal oxide-based conductometric sensors can be integrated with conventional portable integrated electronics and wearable electronics/devices that renders them as portable devices that can be worn if necessary.

In fact, the oxygen-deficient metal oxide-based conductometric sensors can be fabricated on any type of insulating and plastic substrate and still retain selectivity for targeted biomarkers or bioanalytes. Furthermore, these sensors are reusable which further enhances the cost benefit.

### REFERENCES

[1] J. Wang, Z. Wang, B. Huang, Y. Ma, Y. Liu, X. Qin, X. Zhang, Y. Dai, ACS Appl Mater Interfaces 2012, 4, 4024.
[2] M. Chen, X. Wang, Y. H. Yu, Z. L. Pei, X. D. Bai, C. Sun, R. F. Huang, L. S. Wen, Appl. Surf. Sci. 2000, 158, 134.
[3] D. Ehre, H. Cohen, V. Lyahovitskaya, I. Lubomirsky, Physical Review B 2008, 77.
[4] P. V. Nagarkar, P. C. Searson, F. D. Gealy, Journal of Applied Physics 1991, 69, 459.
[5] H. Nili, S. Walia, A. E. Kandjani, R. Ramanathan, P. Gutruf, T. Ahmed, S. Balendhran, V. Bansal, D. B. Strukov, O. Kavehei, M. Bhaskaran, S. Sriram, Adv. Funct. Mater. 2015, 25, 3172.
[6] S. Stille, C. Lenser, R. Dittmann, A. Koehl, I. Krug, R. Muenstermann, J. Perlich, C. M. Schneider, U. Klemradt, R. Waser, Appl. Phys. Lett. 2012, 100, 223503.
[7] H. L. Cai, X. S. Wu, J. Gao, Chem. Phys. Lett. 2009, 467, 313.
[8] P. A. W. v. d. Heide, Q. D. Jiang, Y. S. Kim, J. W. Rabalais, Surf Sci. 2001, 473, 59.
[9] H. Nili, S. Walia, A. E. Kandjani, R. Ramanathan, P. Gutruf, T. Ahmed, S. Balendhran, V. Bansal, D. B. Strukov, O. Kavehei, M. Bhaskaran, S. Sriram, Advanced Functional Materials 2015.
[10] Lorey, S. et al. “Novel Ubiquitin-Derived High Affinity Binding Proteins with Tumor Targeting Properties”, J Bio Chem. (2014); 289(12): pp. 8493-507
[11] Feldwisch, J. and Tolmachev, V. “Engineering of affibody molecules for therapy and diagnostics”, Methods in Molecular Biology (2012); 899: pp 103-126.
[12] Hoffmann T. et al. “Structure-function studies of an engineered scaffold protein derived from stefin A. I: Development of the SQM variant” (2010) Protein Engineering, Design & Selection: PEDS, 23 (5): pp 403-13.
[13] Mouratou et al. “Remodeling a DNA-binding protein as a specific in vivo inhibitor of bacterial secretin PulD” (2007) PNAS. November 13; 104(46): 17983-17988.
[14] US 2010/0305304 A1.
[15] Nucleic Acids Research, Volume 32, Issue suppl_1, 1 Jan. 2004, pp. 95-100, https://doi.org/10.1093/nar/gkh094.
[16] Reverdatto S. et al. “Peptide Aptamers: Development and Applications”, Curr Top Med Chem. (2015), 15(12): pp 1082-1101.
[17] Gebauer, M. and Skerra, A., “Anticalins: small engineered binding proteins based on the lipocalin scaffold”, Methods Enzymology, 503 (2012), pp. 157-188.
[18] Richter, A. et al. “Anticalins: Exploiting a non-Ig scaffold with hypervariable loops for the engineering of binding proteins”, FEBS Letters (2014), 588(2): pp 213-218.
[19] Parmeggiani F et al. “Designed armadillo repeat proteins as general peptide-binding scaffolds: consensus design and computational optimization of the hydrophobic core.” J Mol Biol. (2008), 376(5): pp 1282-304.
[20] Stumpp M T, et al. “DARPins: a new generation of protein therapeutics”, Drug Discovery Today (2008); 13(15-16): pp 695-701.
[21] Pluckthurn A “Designed ankyrin repeat proteins (DARPins): binding proteins for research, diagnostics, and therapy”, Annual Reviews in Pharmacology and Toxicology (2015); 55: pp 489-511.
[22] Moore, S. et al., Protein Engineering for Therapeutics, Part B—Chapter 9, Methods in Enzymology (2012), 503; pp. 223-251.
[23] Park S H et al. “Isolation and Characterization of a Monobody with a Fibronectin Domain III Scaffold That Specifically Binds EphA2”, PLoS One (2015) 10(7) doi: 10.1002/pro.3148.
[24] Lipovsek, D. “Adnectins: engineered target-binding protein therapeutics”, Protein Engineering Design & Selection: PEDS, (2011); 24(1-2):3-9.
[25] Howard and Kaser, Making and Using Antibodies: A Practical Handbook, CRC Press, 2007.
[26] Li, B. et al. “In vitro affinity maturation of a natural human antibody overcomes a barrier to in vivo affinity maturation. MAbs. (2014) 6(2): pp. 437-45.
[27] Nuttall, S., “Overview and discovery of IgNARs and generation of VNARs”, Methods Mol Biol, (2012) 911: pp. 27-36.
[28] Vincke C et al. “Generation of single-domain antibody fragments derived from camelids and generation of manifold constructs”, Methods Mol Biol. (2012) 907: pp. 145-76.
[29] Sjögren, J. et al. “Generating and Purifying Fab Fragments from Human and Mouse IgG Using the Bacterial Enzymes IdeS, SpeB and Kgp.”, Methods Mol Biol. (2017); 1535: pp. 319-329.
[30] Cho Sung Bo et al., Label-free and direct detection of C-reactive protein using reduced graphene oxide-nanoparticle hybrid impedimetric sensor, South Korean Patent 101774891 B1 (2016).
[31] Yang Ting et al., Electrochemical immunosensor for directly detecting IL-6 (interleukin-6) antigen and application thereof, Chinese Patent 102706939 A (2012).
[32] James H. Silver, Sensors for detecting substances indicative of stroke, ischeia, infection or inflammation, U.S. Pat. No. 8,133,698 B2 (2012).

Definitions

Whenever a range is given in the specification, for example, a temperature range, a time range, or concentration range, all intermediate ranges and subranges, as well as all individual values included in the ranges given are intended to be included in the disclosure. It will be understood that any subranges or individual values in a range or subrange that are included in the description herein can be excluded from the claims herein.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

While the invention has been described in conjunction with a limited number of embodiments, it will be appreciated by those skilled in the art that many alternatives, modifications and variations in light of the foregoing description are possible. Accordingly, the present invention is intended to embrace all such alternatives, modifications and variations as may fall within the spirit and scope of the invention as disclosed.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

The claims defining the scope of the invention are as follows:

1. A sensor for detecting a bioanalyte, comprising:
a substrate;
a pair of terminal electrodes disposed on the substrate in mutually spaced apart and opposing relation; and
a non-insulating sensing element applied to a surface of the substrate, between and in electrical contact with the pair of terminal electrodes wherein the sensing element provides a conduction path between the terminal electrodes, wherein the sensing element comprises an oxygen-deficient metal oxide layer and a bioanalyte binding site, and wherein when a voltage is applied across the sensor, an electrical signal is generated that is proportional to a change in conductance of the sensing element corresponding to binding of a bioanalyte to the bioanalyte binding site.

2. A sensor according to claim 1, wherein the oxygen-deficient metal oxide layer is formed from a metal oxide selected from the group consisting of zinc oxide (ZnO), strontium titanium oxide (STO), tin oxide and titanium dioxide.

3. A sensor according to claim 1 or claim 2, wherein the oxygen-deficient metal oxide layer has a thickness that falls within a range of about 50 nm to about 200 μm.

4. A sensor according to any one of claims 1 to 3, wherein the oxygen-deficient metal oxide layer is applied to the substrate surface by a technique selected from the group consisting of reactive sputtering, physical vapour deposition (PVD), chemical vapour deposition (CVD), metal organic chemical vapour deposition (MOCVD), pulsed laser deposition (PLD) and molecular beam epitaxy (MBE).

5. A sensor according to any one of claims 1 to 4, wherein the bioanalyte binding site is anchored to the oxygen-deficient metal oxide layer via an intermediate layer physically or chemically adsorbed to the oxygen-deficient metal oxide layer.

6. A sensor according to claim 5, wherein the intermediate layer is produced by silanization of the oxygen-deficient metal oxide layer with a silanizing agent having a terminal functionality that is selected from the group consisting of an epoxy group, a thiol group, an amino group, a carboxy group and a hydroxy group.

7. A sensor according to claim 6, wherein the silanizing agent is selected from the group consisting of (3-glycidyloxypropyl)trimethoxysilane, (3-mercaptopropyl)trimethoxysilane (MTS), (3-aminopropyl)triethoxysilane (APTES), and N-(2-aminoethyl)-3-aminopropyl-trimethoxysilane (AEAPTS).

8. A sensor according to any one of claims 1 to 7, wherein the oxygen-deficient metal oxide layer has a conductance that falls within a range of about 0.08 siemens/m² to about 0.6 siemens/m².

9. A sensor according to any one of claims 1 to 8, wherein the bioanalyte binding site is a biomolecule.

10. A sensor according to claim 9, wherein the biomolecule is a protein, a peptide, a lipo-peptide, a protein-binding carbohydrate or a protein-binding ligand.

11. A sensor according to claim 9, wherein the biomolecule is a capture protein.

12. A sensor according to claim 11, wherein the capture protein is a protein-binding scaffold, a T-cell receptor, a binding-fragment of a TCR, a variable lymphocyte receptor, an antibody and/or a binding-fragment of an antibody.

13. A sensor according to claim 12, wherein the protein-binding scaffold is selected from the group consisting of: Adnectins, Affilins, Affibodies, Affimer molecules, Affitins, Alphabodies, Aptamers, Anticalins, Armadillo repeat protein-based scaffolds, Atrimers, Avimers, Designed Ankyrin Repeat Proteins (DARPins), Fynomers, Inhibitor Cystine Knot (ICK) scaffolds, Kunitz Domain peptides, Monobodies and/or Nanofitins.

14. A sensor according to claim 12, wherein the binding-fragment of an antibody includes a Fab, (Fab′)₂, Fab′, single-chain variable fragment (scFv), di- and tri-scFvs, single domain antibodies (sdAb), Diabodies or a fusion protein including a binding-domain of an antibody.

15. A sensor according to any one of claims 1 to 14, wherein the bioanalyte binding site binds interleukin-6 (IL-6).

16. A sensor according to any one of claims 1 to 14, wherein the bioanalyte binding site binds C-reactive protein (CRP).

17. A sensor according to any one of claims 1 to 16, wherein the substrate is manufactured from a material selected from the group consisting of a silicon wafer, a polymer, a glass and a ceramic.

18. A sensor according to claim 17, wherein the polymer is selected from the group consisting of polydimethylsiloxane (PDMS), polyimide (PI) and polyethylene naphthalate (PEN).

19. A sensor according to claim 17, wherein the ceramic is selected from the group consisting of aluminium oxide (Al₂O₃), sapphire and silicon nitride (Si₃N₄).

20. A method for detecting a bioanalyte, the method comprising the steps of:
a) contacting a sensing element of a sensor according to any one of claims 1 to 19 with a sample solution comprising a bioanalyte;
b) applying a voltage across the sensor; and
c) detecting an electrical signal generated that is proportional to a change in conductance corresponding to detection of the bioanalyte upon binding of the bioanalyte to the bioanalyte binding site.

21. A method according to claim 20, wherein the bioanalyte binding site is a biomolecule.

22. A method according to claim 20 or claim 21, wherein the bioanalyte binding site binds interleukin-6 (IL-6).

23. A method according to claim 22, wherein the change in conductance detected in a sample solution with a concentration of IL-6 of 4 femtomolar is about 9.2%.

24. A method according to claim 20 or claim 21, wherein the bioanalyte binding site binds C-reactive protein (CRP).

25. A method according to claim 24, wherein the change in conductance detected in a sample solution with a concentration of CRP of 13 femtomolar is about

26. A method of fabricating a sensor for detecting a bioanalyte, the method comprising the steps of:
providing a substrate;
depositing a pair of terminal electrodes on the substrate in mutually spaced apart and opposing relation; and
applying a non-insulating sensing element in the form of an oxygen-deficient metal oxide layer coated with a bioanalyte binding site, between and in electrical contact with the pair of terminal electrodes wherein the sensing element provides a conduction path between the terminal electrodes, wherein the bioanalyte binding site is selective toward detection of a bioanalyte upon binding of the bioanalyte to the bioanalyte binding site.

27. A method according to claim 26, wherein the oxygen-deficient metal oxide layer is formed from a metal oxide selected from the group consisting of zinc oxide (ZnO), strontium titanium oxide (STO), tin oxide and titanium dioxide.

28. A method according to claim 26 or claim 27, wherein the oxygen-deficient metal oxide layer has a thickness that falls within a range of about 50 nm to about 200 μm.

29. A method according to any one of claims 26 to 28, wherein the oxygen-deficient metal oxide layer is applied to the substrate surface by a technique selected from the group consisting of reactive sputtering, physical vapour deposition (PVD), chemical vapour deposition (CVD), metal organic chemical vapour deposition (MOCVD), pulsed laser deposition (PLD) and molecular beam epitaxy (MBE).

30. A method according to any one of claims 26 to 28, further comprising the step of:
physically or chemically adsorbing an intermediate layer to the oxygen-deficient metal oxide layer for anchoring the bioanalyte binding site to the oxygen-deficient metal oxide layer.

31. A method according to claim 30, wherein the intermediate layer is produced by silanization of the oxygen-deficient metal oxide layer with a silanizing agent having a terminal functionality that is selected from the group consisting of an epoxy group, a thiol group, an amino group, a carboxy group and a hydroxy group.

32. A method according to claim 31, wherein the silanizing agent is selected from the group consisting of (3-glycidyloxypropyl)trimethoxysilane, (3-mercaptopropyl)trimethoxysilane (MTS), (3-aminopropyl)triethoxysilane (APTES), and N-(2-aminoethyl)-3-aminopropyl-trimethoxysilane (AEAPTS).
